(19) United States
(12) Patent Application Publication
(10) Pub. No.: US 2018/0092572 A1
(43) Pub. Date: Apr. 5, 2018

(54) Gathering and Analyzing Kinetic and Kinematic Movement Data

(71) Applicant: ArthroKinetic Institute, LLC, Santa Barbara, CA (US)

(72) Inventors: Eric Sanchez, Santa Barbara, CA (US); Maury Hayashida, Santa Barbara, CA (US); Daniel Price, Grass Valley, CA (US); Daniel deLaveaga, Reno, NV (US)

(21) Appl. No.: 15/724,099

(22) Filed: Oct. 3, 2017

Related U.S. Application Data
(60) Provisional application No. 62/529,306, filed on Jul. 6, 2017.
(60) Provisional application No. 62/457,766, filed on Feb. 10, 2017.
(60) Provisional application No. 62/455,456, filed on Feb. 6, 2017.
(60) Provisional application No. 62/442,328, filed on Jan. 4, 2017.
(60) Provisional application No. 62/404,161, filed on Oct. 4, 2016.

(51) Int. Cl. A61B 5/11; A43B 7/18; A63B 24/00; A63B 69/00; A63B 71/06; A43B 3/00; A61B 5/00; H04Q 9/00

(52) U.S. Cl. CPC A61B 5/112; G06F 19/322; A63B 24/0062; A63B 69/00; A63B 71/0622; A43B 3/0005; A61B 5/0004; A61B 5/6807; A61B 5/4836; A61B 5/7246; A61B 5/742; H04Q 9/00; A63B 2225/50; A63B 2220/40; A63B 2220/803; A63B 2220/89; A63B 2071/0625; A63B 2071/0655; A61B 2562/0219; A61B 2562/0223; A43B 7/18

(57) ABSTRACT

Systems and methods disclosed herein include receiving first data sent over a first wireless communication channel from a first inertial sensor positioned in or on a first shoe worn by a user, wherein the first data are generated when the user performs a fundamental movement; identifying a first phase of the fundamental movement by finding a match, within a first tolerance, between a portion of the first data and data characteristic of the first phase; comparing a feature from the portion of the first data to a corresponding feature from a pre-established signature associated with the first phase; and when the comparison yields a result that falls outside a pre-established threshold range, displaying an indication that the feature from the portion of the first data is uncharacteristic.

### CROSS REFERENCE TO RELATED APPLICATIONS

This application claims priority to U.S. provisional application 62/404,161 filed Oct. 4, 2016, U.S. provisional application 62/442,328 filed Jan. 4, 2017, U.S. provisional application 62/455,456 filed on Feb. 6, 2017, U.S. provisional application 62/457,766 filed on Feb. 10, 2017, and U.S. provisional application 62/529,306 filed on Jul. 6, 2017, each of which is hereby incorporated by reference, as if set forth in full in this specification

### FIELD OF THE DISCLOSURE

Various embodiments described herein relate generally to the field of generating and analyzing data characterizing user movement, and in particular to methods and systems that facilitate such data generation and analysis using wearable motion sensors and data processing to yield diagnostically useful indications of movement and movement quality.

### BACKGROUND

Body movement is generally achieved through a complex and coordinated interaction between bones, muscles, ligaments, and joints within the body's musculoskeletal system. Any injury to, or lesion in, any part of the musculoskeletal system, whether obvious symptoms exist yet or not, can change the mechanical interaction causing faulty body movement, and, if left unchecked or untreated, cause longer term problems, such as degradation, instability, disability of movement, and/or loss of performance opportunities. Even at relatively early stages of injury or disease, specific features of particular movements may change. Observing and understanding those changes may yield information that is of diagnostic significance to an individual user or to medical professionals consulted by the user. In some cases, quick access to such information may allow the user to make appropriate real-time adjustments to his/her movements, possibly with the involvement of orthotic devices.

### BRIEF DESCRIPTION OF THE DRAWINGS

Features, aspects, and advantages of the presently disclosed technology may be better understood with regard to the following description, appended claims, and accompanying figures.

FIG. 1 shows a functional block diagram of a sensor device according to some embodiments of the disclosure.

FIG. 2 shows a functional block diagram of a processing device according to some embodiments of the disclosure.

FIG. 3 illustrates the operation of sensor and processing devices according to some embodiments of the disclosure.

FIG. 4 shows bottom-up and side views of a sensor device worn according to some embodiments of the disclosure.

FIG. 5 shows an example method according to some embodiments of the disclosure.

FIG. 6A shows data obtained from an accelerometer sensor in one embodiment, where the user is walking.

FIG. 6B shows data obtained from a gyrometer sensor in one embodiment, where the user is walking.

FIG. 6C shows data obtained from a magnetometer sensor in one embodiment, where the user is walking.

FIG. 7A shows data obtained from an accelerometer sensor in one embodiment, where the user is running.

FIG. 7B shows data obtained from a gyrometer sensor in one embodiment, where the user is running.

FIG. 8A shows data obtained from an accelerometer sensor in one embodiment, where the user is single leg jumping.

FIG. 8B shows data obtained from a gyrometer sensor in one embodiment, where the user is single leg jumping.

FIG. 9A shows data obtained from an accelerometer sensor in one embodiment, where the user is squatting.

FIG. 9B shows data obtained from a gyrometer sensor in one embodiment, where the user is squatting.

FIG. 10A shows data obtained from an accelerometer sensor in one embodiment, where the user is changing direction.

FIG. 10B shows data obtained from a gyrometer sensor in one embodiment, where the user is changing direction.

FIG. 10C shows data obtained from a magnetometer sensor in one embodiment, where the user is changing direction.

FIG. 11A shows data obtained from an accelerometer sensor in one embodiment, where the user is limping.

FIG. 11B shows data obtained from a gyrometer sensor in one embodiment, where the user is limping.

FIG. 12A shows data obtained from an accelerometer sensor in one embodiment, where the user is walking too slowly.

FIG. 12B shows data obtained from a gyrometer sensor in one embodiment, where the user is walking too slowly.

FIG. 13A shows data obtained from an accelerometer sensor in one embodiment, during the heel strike phase.

FIG. 13B shows data obtained from a gyrometer sensor in one embodiment, during the heel strike phase.

FIG. 14A shows data obtained from an accelerometer sensor in one embodiment, during the mid-stance phase.

FIG. 14B shows data obtained from a gyrometer sensor in one embodiment, during the mid-stance phase.

FIG. 15A shows data obtained from an accelerometer sensor in one embodiment, during the terminal stance phase.

FIG. 15B shows data obtained from a gyrometer sensor in one embodiment, during the terminal stance phase.

FIG. 16A shows data obtained from an accelerometer sensor in one embodiment, where the user has a heavy heel strike.

FIG. 16B shows data obtained from a gyrometer sensor in one embodiment, where the user has a heavy heel strike.

FIG. 16C shows data obtained from a magnetometer sensor in one embodiment, where the user has a heavy heel strike.

FIG. 17A shows data obtained from an accelerometer sensor in one embodiment, where the user has an abnormally long toe-off phase.

FIG. 17B shows data obtained from a gyrometer sensor in one embodiment, where the user has an abnormally long toe-off phase.

FIG. 17C shows data obtained from a magnetometer sensor in one embodiment, where the user has an abnormally long toe-off phase. The figures are for purposes of illustrating example embodiments, but it is understood that the inventions are not limited to the arrangements and instrumentality shown in the drawings. In the figures, identical reference numbers identify at least generally similar elements.

### DETAILED DESCRIPTION

### I. Overview

Tracking changes in specific movement features over time may provide, among other things, a useful indication of the progress of injury or disease, or of the efficacy of any remedial measures taken by the user and/or medical professionals. Such indications of progress may be of value to, among others, the user's medical insurer, to those making clinical decisions, and to help users who may enjoy good health, and are free of injury, but are interested in improving their performance in recreational or professional sporting activities.

There is therefore an identified need for methods and systems that generate data indicative of a user's kinetic and/or kinematic movement. Kinetics generally refers to forces involved with movements; kinematics generally refers to the movements of body parts in relation to each other. There is a further identified need to analyze the data to yield information of value to the user and/or to other authorized entities. Such methods and systems would ideally gather data as unobtrusively as possible, making minimal demands on the user, and analyze the data to provide feedback either in real-time or after storage for review at a later time, indicating to the user whether features of the movement fall within expected norms.

One may envisage many challenges that embodiments described herein address. Several include, for example:

1) There is a technical complexity to more effectively gather the right data, analyze and interpret the data, and provide appropriate and credible output to the intended recipient. For instance, video may be used to analyze kinetic and/or kinematic movement, but such analysis typically requires expensive equipment, careful setup of a controlled environment, and a lot of effort and expertise. Using this example, a specialized physician might use video equipment in their clinic or office to observe and analyze a person's gait as part of medical evaluation and/or treatment. However, for instance, it is less likely that a store that specializes in running shoes would afford the equipment and/or have the expertise to credibly evaluate the results.

2) There is a technical complexity to gather the right data using less costly and/or cumbersome technology. Such as in the example of the clinic above, a specialized physician may use all sorts of expensive equipment to gather data. Embodiments herein aim to understand and/or infer from the sensor-provided data a kinetic and/or kinematic movement other than the movement localized in the area of the sensor(s), and/or identify a problem with the movement. These embodiments provide interpretation of aspects of the data generated by the sensor(s) (including granularity of the data and noise introduced into the data from wearing the sensor(s)).

3) There is a technical complexity in interpreting the data. The results of the data analysis may call for a deep understanding of kinetic and/or kinematic movement. Some of these embodiments provide a more simplified presentation that can be understood, for example, by a layperson. This presentation may include an identification of a user's movement, an analysis of the movement, and/or corrective action to improve the movement. Further, there may be benefit to presenting the results directly to the user on a personal device (possibly even during the movement) rather than requiring a trained professional to evaluate the results and present them to the user (most often at a later time). As such, in some instances, the user has access to deep levels of analysis without having to be in a doctor's office or other specialized facility, receiving accurate diagnosis of the issue(s) of interest without needing a doctor or other professional to be present.

4) There is a technical complexity in data gathering, analyzing and interpreting the data, and providing output in an environment outside of a clinic, lab, or office. Many movements performed in “normal” life situations may not be exactly repeatable in clinical settings such as a lab or doctor's office. Similarly, movements performed in a lab or office situation may be different—possibly less natural—than those in daily life; this is analogous to “white coat hypertension” which is a documented phenomenon in which patients exhibit higher than normal blood pressure readings in a clinical setting compared to other, more normal settings. In both cases, the analysis of lab-generated data can lead to artificial results. Embodiments herein can gather and analyze data performed in relatively normal, real life situations.

5) There is a technical complexity in identifying problems or issues prior to experiencing symptoms. As in the clinic example above, most if not all, patients visit a doctor that specializes in kinematic movement when symptoms arise or exist. Some embodiments herein can help identify problems or issues before symptoms are presented, and in some instances, provide a corrective action.

6) There is a technical complexity in identifying a performance improvement for complex movements. For example, a typical performance measurement for a football running back may be his/her running speed and/or stride length, which can be measured using a timing apparatus and a measured distance. Often, a diminishing stride length will decrease the speed and performance, and it would be natural to suggest to the running back to increase their stride length for better performance. However, if there was a mechanism to understand the running back was landing harder on one foot compared to the other foot, then increasing the stride length may not help the performance, and instead may cause or exacerbate an injury.

7) There is a technical complexity in understanding the quality of care for a patient who is receiving treatment. For example, an insurance company may want to understand the recovery progress for a patient who is seeing a doctor or physical therapist after hip replacement surgery. Some embodiments herein can be used to indicate the recovery progress without requiring a detailed analysis and documentation by the doctor or physical therapist.

### II. Example Sensor Devices

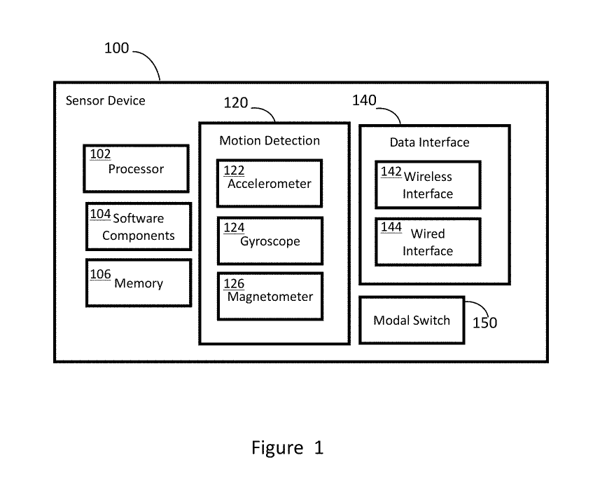

FIG. 1 shows a functional block diagram of an example sensor device 100 according to one embodiment. Sensor device 100 includes one or more processors 102, software components 104, memory 106, a motion detection sensor block 120, a data interface 140, and a modal switch 150. Motion detection sensor block 120 may include one or more combinations of distinct types of inertial sensors shown in FIG. 1—accelerometer 122, gyrometer (used herein interchangeably with “gyroscope”) 124, and magnetometer 126. Data interface 140 may include one or both wireless (142) and wired (144) interfaces. Sensor device 100 may take many different form factors depending on the application.

The processor 102 may include one or more general-purpose and/or special-purpose processors and/or microprocessors that are configured to perform various operations of a computing device (e.g., a central processing unit). The memory 106 may include a non-transitory computer-readable medium configured to store instructions executable by the one or more processors 102. For instance, the memory 106 may be data storage that can be loaded with one or more of the software components 104, executable by the one or more processors 102 to achieve certain functions. In one example, the functions may involve collecting inertial data from the one or more sensors 122-126 and transmitting the inertial data to another device over the data interface 140.

As noted above, the motion detection sensor block 120 includes one or more inertial sensors such as, for example, accelerometer 122, gyrometer 124, and magnetometer 126. Considering a single axis for simplicity, an accelerometer measures linear acceleration along that axis, from which force can be derived, a gyrometer measures angular velocity about that axis, from which rotational motion direction can be derived, and a magnetometer measures magnetic flux density along that axis, from which orientation with respect to the earth's surface can be derived. Each sensor 122, 124, and 126 may have multi-axis (either 2-axis or more typically 3-axis) sensing capability, and each sensor may be able to collect sensor data simultaneously. For example, the accelerometer 122 may be able to collect acceleration force data at the same time the gyrometer 124 collects rotational motion data. Similarly, the magnetometer 126 may be able to collect orientation data at the same time the accelerometer 122 collects acceleration force data. As may be understood by one having ordinary skill in the art upon reading this disclosure, other combinations exist.

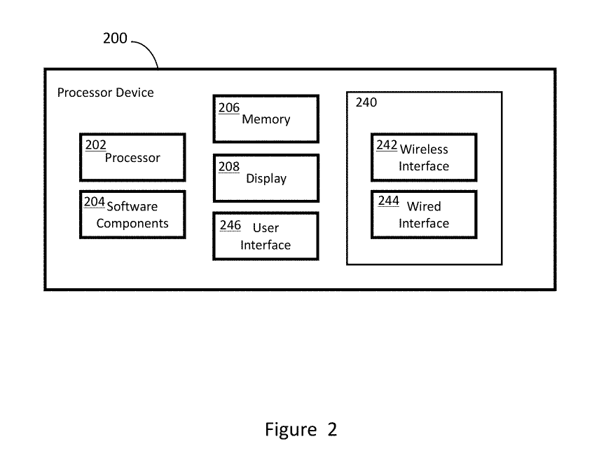

The data interface may be configured to facilitate a data flow between the sensor device 100 and one or more other devices, including but not limited to data to/from other sensor devices 100 or processing devices 200 (shown and discussed in relation to FIG. 2). As shown in FIG. 1, the data interface 140 may include wireless interface(s) 142 and wired interface(s) 144. The wireless interface(s) 142 may provide data interface functions for the sensor device 100 to wirelessly communicate with other devices (e.g., other sensor device(s), processing device(s), etc.) in accordance with a communication protocol (e.g., a wireless standard including, for instance, IEEE 802.15, 802.11a, 802.11b, 802.11g, 802.11n, 802.11ac, 4G mobile communication standard, and so on). The wired interface(s) 144 may provide data interface functions for the sensor device 100 to communicate over a wired connection with other devices in accordance with a communication protocol (e.g., USB 2.0, 3.x, micro-USB, Lightning® by Apple®, IEEE 802.3, etc.). While the data interface 140 shown in FIG. 1 includes both wireless interface(s) 142 and wired interface(s) 144, data interface 140 may in some embodiments include only wireless interface(s) 142 or only wired interface(s) 144.

The modal switch 150 may be configured to toggle the operation of the sensor device 100 between operating modes. For example, some example modes may include programming mode, diagnostic mode, and operational mode.

### III. Example Processing Devices

FIG. 2 shows a functional block diagram of an example processing device 200 according to one embodiment. Processing device 200 includes one or more processors 202, software components 204, memory 206, a display 208, a data interface 240 which may include a wireless interface 242 and/or a wired interface 244, and a user interface 246. For purposes of illustration only, processing device 200 may be a device, such as for example, a mobile phone, tablet, laptop, connected watch, smart glasses, or other connected portable and/or wearable device. In other examples, processing device 200 may be less portable, such as a desktop PC/Mac computer or a server device.

Processor 202 may include one or more general-purpose and/or special-purpose processors and/or microprocessors that are configured to perform various operations of a computing device (e.g., a central processing unit). Memory 206 may include a non-transitory computer-readable medium configured to store instructions executable by the one or more processors 202. For instance, memory 206 may be data storage that can be loaded with one or more of the software components 204, executable by the one or more processors 202 to achieve certain functions.

The data interface 240 may be configured to facilitate a data flow between the processing device 200 and one or more other devices, including but not limited to data to/from the sensor device 100 or other networked devices. The data interface 240 may include wireless interface(s) 242 and wired interface(s) 244. Wireless interface(s) 242 may provide data interface functions for the processing device 200 to wirelessly communicate with other devices (e.g., other sensor device(s), processing device(s), etc.) in accordance with a communication protocol (e.g., a wireless standard including, for instance, IEEE 802.15, 802.11a, 802.11b, 802.11g, 802.11n, 802.11ac, 4G mobile communication standard, and so on). The wired interface(s) 244 may provide data interface functions for the processing device 200 to communicate over a wired connection with other devices in accordance with a communication protocol (e.g., USB 2.0, 3.x, micro-USB, Lightning by Apple, IEEE 802.3, etc.). While the data interface 240 shown in FIG. 2 includes both wireless interface(s) 242 and wired interface(s) 244, data interface 240 may in some embodiments include only wireless interface(s) 242 or only wired interface(s) 244.

User interface 246 may generally facilitate user interaction with processing device 200 and control of sensor device 100. Specifically, user interface 246 may be configured to detect user inputs and/or provide feedback to a user, such as audio, visual, audiovisual, and/or tactile feedback. As such, user interface 246 may be or include one or more input interfaces, such as mechanical buttons, “soft” buttons, dials, touch-screens, etc. In example implementations, user interface 246 may take the form of a graphical user interface configured with input and output capabilities. As may be understood by one having ordinary skill in the art upon reading this disclosure, other examples are also possible.

Display 208 may generally facilitate the display of information. For example, some results of the analysis, notifications, suggested movement corrections, etc. may be displayed to the user via display 208.

### IV. Example Operating Environment

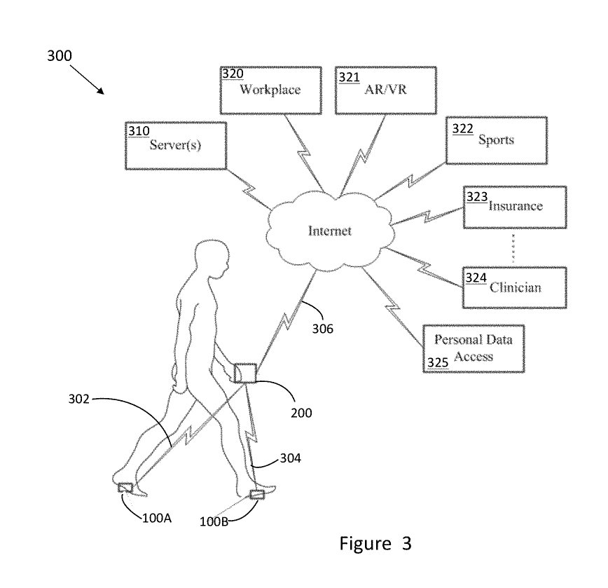

FIG. 3 shows an example configuration of an inertial sensor system 300 in which one or more embodiments disclosed herein may be practiced or implemented. Inertial sensor system 300 includes a user wearing two sensor devices 100A and 100B and a processing device 200 that may be held in the user's hand or worn, for example on the user's wrist. Note that sensor devices 100A and 100B may be the same as sensor device 100 of FIG. 1, and processing device 200 may be the same as processing device 200 of FIG. 2. Additionally, it is understood that in some embodiments the user may wear only a single sensor device and in other embodiments the user may wear more than two sensor devices. The sensor devices 100A and 100B are shown transmitting data via paths 302 and 304, respectively, to processing device 200. Additionally, processing device 200 is shown communicating through path 306 via a network (e.g., a local area network or LAN, or the Internet) to one or more servers 310 or other computing devices 320-325 that are accessible by entities authorized by the user.

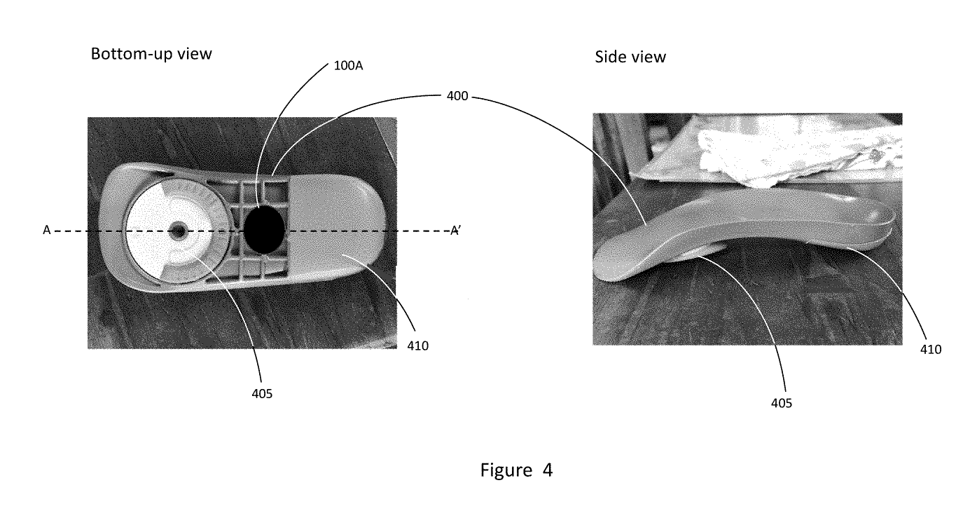

In the example embodiment shown in FIG. 3, each sensor device 100A,100B is placed in or around the sole of a corresponding shoe or in a corresponding orthotic shoe insole (also called an “orthotic shoe insert”) placed in the shoe. FIG. 4 illustrates one instance, in which sensor device 100A and/or 100B is embedded into insole 400 between stiffness-setting dial 405 and heel 410. In other instances, the size and shape of sensor device 100A and/or 100B may differ from those shown, and the position of the device may also differ; for example, it may be positioned asymmetrically rather than symmetrically with respect to the central axis AA' of the insole.

In some embodiments, sensor device 100A and/or 100B may be accessible by the user to manually turn the sensor device on or off, while in other cases the operation of the sensor device may include a “sleep mode” where the sensor device remains in a dormant (e.g., low-power) state but is automatically turned to a full “on” state by motion. In some embodiments, the sensor device may turn off or return to sleep mode after a predetermined time interval, if no motion is detected during that interval.

In some embodiments, sensor device 100A and/or 100B may be accessible by the user so that, for example, a battery may be replaced, or so that the entire sensor device may be replaced by another sensor device. In some embodiments, the battery may not be accessible and the insole or insert would be discarded once the battery is depleted. Other examples may readily be envisaged.

The embodiments of FIGS. 3 and 4 involve the positioning of sensor devices in insoles or shoe inserts, delivering data directly indicative of foot and ankle movement, but the data provided by such sensor devices may also be used to infer facts about movement at other joints of the body, such as, for example, the knee, hip or pelvis. Other embodiments may involve similar sensor devices positioned on or near other parts of the body, such as the knee, hip, or pelvis, so that data gathered and delivered to the processing device may be more directly indicative of the biomechanics of that body part.

In some embodiments, the analysis of data by the mobile processing device 200 includes comparing one or more features of the measured data with features of a pre-established “signature” or “signatures.” One or more pre-established “signatures” may be stored on the mobile device, on a server in the cloud, in a local network server, or a combination of these. Based on the analysis, the mobile device in this instance may provide, for example, (1) an indication of the fundamental movement that the user is performing or has performed (e.g., walking, running, and other kinds of fundamental movements), (2) an indication whether the user wearing the insert is moving incorrectly (or correctly, if so desired); and/or (3) an indication that the insert itself needs adjustment. Methods by which a “signature” is established are described below in the “Signature Establishment” section.

Referring back to FIGS. 1-3, some additional aspects of data collection and communication will now be described. In one embodiment, each sensor device independently transmits data over the wireless data interface 142 to the processing device 200 using wireless technology such as, for example, WiFi, Bluetooth, Bluetooth Low Energy (BTLE), NFC, etc. Communication may be direct (point-to-point) between each sensor device 100 and the processing device 200 or communication may be routed through an Access Point or other networking relay device that can be used to propagate data packets from one networking device to another. The data may be sent periodically during a movement/exercise or when a movement/exercise is complete. The data transmission may also be contingent upon having a good network connection and/or battery life. For example, data may be sent upon detection of the completion of one or more movement/exercises among a series of movements/exercises (i.e. after one or more laps, miles, reps, etc. are detected) or periodically at regular time intervals (e.g., 1, 5, 10 sec).

In another embodiment, each sensor device 100 independently transmits data to a network server 310 or other computing device 320-325 through a connected device for processing. For example, the data are transmitted wirelessly to a first connected device (e.g., mobile phone, tablet, PC/Mac, connected watch, glasses or other connected wearable device) that may do some preliminary processing before sending to the network server 310 for processing. The communication between the first connected device and the server 310 or other computing device 320-325 may use wired or wireless technologies and typical networking protocols.

Throughout this disclosure, the term “fundamental movement” is understood to be any one of the following movements (or substantially similar movements): walking, running, single-leg jumping, double-leg jumping, skip and hop, squatting, partial squatting, and shuffle direction change. Each of these fundamental movements may be considered to include one or more phases, which in turn may be considered to include one or more sub-phases. For the remainder of this disclosure, the term “sub-phase” will be dropped for simplicity, and be understood as being covered by the term “phase”.

### V. Signature Establishment

Signatures representative of fundamental movements, or portions thereof, may be established manually or automatically as described below. While the descriptions refer to “the user”, signatures representative of particular populations or sub-populations of users may also be established based on data gathered from a plurality of corresponding users. The signatures may also represent unique movement features about the user that are relevant to their health and function. The populations may be based on age, height, weight, gender, disability status, stage of recovery from injury, or various other criteria or combinations of criteria. A signature may be a single series of signal magnitude values vs time, or multiple series of signal magnitudes vs time, each corresponding to one of up to three axes for each of the different types of inertial sensor involved.

(1) Manual Setup Procedure. This can be done, for example, at a clinic, at a lab, at the user's home, or any other chosen location. The user inputs (via user interface 246 in FIG. 2, for example) an indication into the processing device (e.g. device 200 in FIG. 2) that the user is going to perform a fundamental movement, such as “walking”. The user then performs the intended movement and corresponding inertial movement data are gathered by the sensor device(s) (e.g., device 100 in FIG. 1), transmitted to the processing device, and used to determine a baseline signature for that movement. The baseline signature is then stored in a signature database held within a data storage area of the processing device, a local network server, and/or a cloud server, as representing “normal” for the user for that fundamental movement. In some cases, one or more phases of a fundamental movement may be identified from the received data, and corresponding data points extracted and stored in the same way, as signatures of the corresponding phases of the fundamental movements. In one embodiment, such identifications may be carried out by a clinician working with data analysis software. In another embodiment, artificial intelligence systems employing machine learning may be used to pick out the data corresponding to phases of interest without direct human intervention.

(2) Automatic Setup Procedure. Without requiring input from the user about which movement is to be performed, data on whatever movements the user performs are captured over time by the processing device, and a baseline signature for any fundamental movement or phase or phases thereof may be determined from the data. The baseline signature may then be stored in a signature database held within a data storage area of the processing device, a local network server, and/or a cloud server, as representing “normal” for the user for that fundamental movement.

As mentioned above, one or more of the established signatures may be generated based on a single user or a small to large population. Once established, a signature may further be refined based on additional information and/or data collection.

As such, a signature may be further personalized to a user based on data collected over time corresponding to the user's movement. In other words, even though in some embodiments a signature may be established based on data from a population, the signature may further be personalized based on a user's own data representing the movement.

### VI. Data Analysis

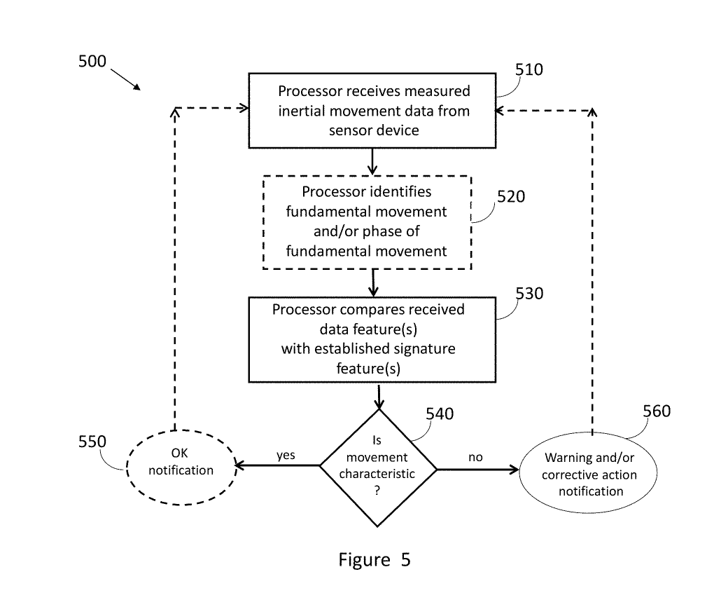

Method 500 in FIG. 5 shows an embodiment of an example method that can be implemented within an operating environment including or involving, for example, one or more sensors 100 of FIG. 1, one or more processing devices 200 of FIG. 2, and/or a system 300 of FIG. 3.

Method 500 may include one or more operations, functions, or actions as illustrated by one or more of blocks 510, 520, 530, 540, 550, and 560. Although the blocks are illustrated in sequential order, these blocks may also be performed in parallel, and/or in a different order than those described herein. Also, the various blocks may be combined into fewer blocks, divided into additional blocks, and/or removed based upon the desired implementation.

In addition, for the method 500 and other processes and methods disclosed herein, the flowchart shows functionality and operation of one possible implementation of some embodiments. In this regard, each block may represent a module, a segment, or a portion of program code, which includes one or more instructions executable by one or more processors for implementing specific logical functions or steps in the process. The program code may be stored on any type of computer readable medium, for example, such as a storage device including a disk or hard drive. The computer readable medium may include non-transitory computer readable medium, for example, such as tangible, non-transitory computer-readable media that stores data for short periods of time like register memory, processor cache and Random Access Memory (RAM). The computer readable medium may also include non-transitory media, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a computer readable storage medium, for example, or a tangible storage device. In addition, for the method 500 and other processes and methods disclosed herein, each block in FIG. 5 may represent circuitry that is wired to perform the specific logical functions in the process.

At block 510, the processor of the processing device (e.g., processing device 200 of FIG. 2) receives data measured by one or more sensor devices containing one or more inertial movement sensors while the user performs a movement. In some cases, optional block 520 is carried out, in which the processor (e.g., of device 200) analyzes the received data to identify the movement carried out by the user as a fundamental movement or as a phase of a fundamental movement. In other cases, the user may identify the movement as a fundamental movement using, for example, user interface 246 as mentioned above, so block 520 is omitted.

At block 530, the processor (e.g., of device 200) compares one or more features of the received data with features of the identified fundamental movement or phase in the signature for that fundamental movement or phase, the signature having been previously established as described in section V above, and stored in the memory of the processing device or in some other database accessible to the processor device.

At block 540, the processor (e.g., of device 200) determines, on the basis of the comparison with the pre-established signature, whether or not the movement carried out by the user may be considered “characteristic”, where the term “characteristic” is defined to mean that a relevant feature of the received data falls within a pre-established range for that feature in the corresponding signature for the identified fundamental movement or phase of fundamental movement.

At optional block 550, if the determination is positive, a notification may be sent to the user, for example by a green light on a display, indicating that all is well. However, if the determination is negative, meaning that the movement is not characteristic, block 560 is carried out, to provide a notification to the user either warning of the incorrectness of the movement and/or recommending corrective action to be taken. In some embodiments, not shown, a warning notification and/or corrective suggestions may be provided even when the movement is considered characteristic, but when either one limit of the pre-established range is very close, or when the trend over time of data collected from the particular user indicates that it may soon be closely approached.

In embodiments where time-based comparisons are made between data from two or more independent sensors, the processing device can be the “arbiter of time”. This can be done, for example, by the processing device assigning a timestamp to each series of data received from each sensor when it is received at the processing device.

### Examples of Identifying Fundamental Movements and/or Phases Thereof

The following examples concern block 520 of FIG. 5.

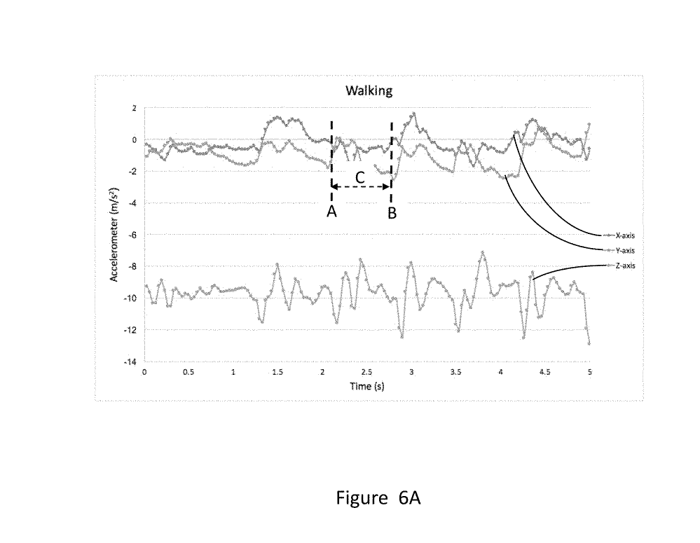

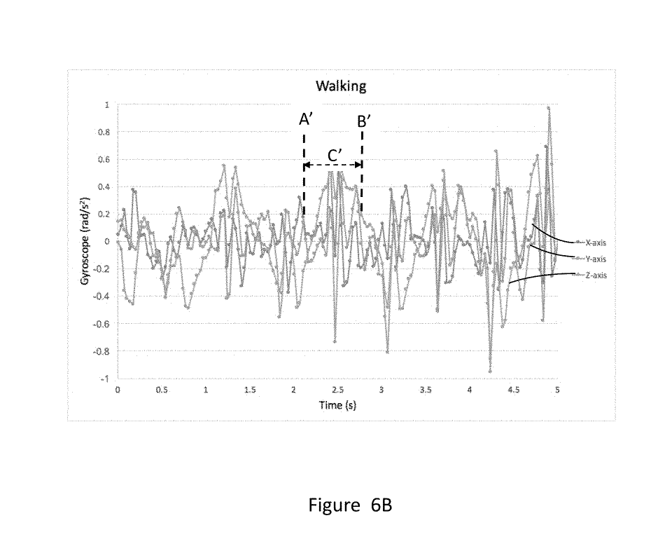

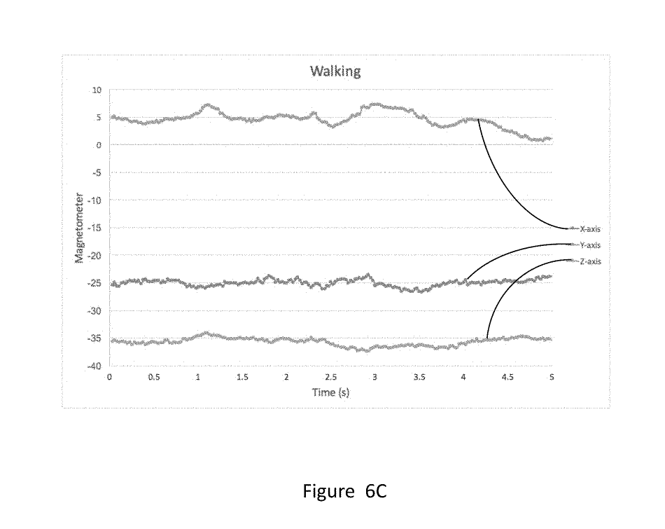

One example embodiment would identify the user movement as the fundamental movement of “walking” by examining the data generated by at least one sensor in each of the user's shoes, and wirelessly transmitted to a processing device (e.g., processing device 200 of FIG. 2), using a technology such as BTLE. For example, in the case where an accelerometer is present in each shoe, ground reaction force data (derived from linear acceleration) are gathered as a function of time, in the form of three series of data points for each foot, one series for each axis. FIG. 6A shows measured three-axis data obtained from an accelerometer in one shoe. Features A correspond to toes lifting off the ground (“toe-off”), that features B correspond to heels striking the ground, and that interval C corresponds to a “swing phase”, during which the foot is off the ground (after toe-off but before the next heel strike). In one embodiment, the processor (e.g., of device 200) can determine that the user is walking by comparing the sensor data received from two sensors (e.g., one corresponding to the left foot and one corresponding to the right foot). In this case, time-based measurements are processed from two sensors, and the processing device, as the time arbiter, determines that the heel strike for one foot occurs before the toe-off for the other foot. In other words, at least one foot is in contact with the ground at any instant in time, which is a defining characteristic of walking.

FIG. 6B shows measured three axis-data obtained from a gyrometer in one shoe, providing data on rotational force (derived from angular rotation about each axis) as a function of time. Features A′, B′, and C′ correlate in time to features A, B, and C in FIG. 6A, indicating toe-off, heel strike, and swing phase respectively. Although not shown in FIG. 6B, a strong peak in magnitude outside a defined norm in the gyrometer axis during the toe-off or heel strike may indicate a risk for a number of injuries to the foot, ankle, knee, hip, etc. For example, this may be an indication of plantar fascitis and arthritis of the knee. Depending on how regular a strong peak in magnitude occurs, and depending on if the peak(s) occurs during the toe-off or heel strike, may indicate different injuries or potential injuries.

FIG. 6C shows measured three axis-data obtained from a magnetometer sensor in one shoe. Although not shown in FIG. 6C, a strong directional change in movement may indicate a loss of balance, intoxication, or some other issue worth recording and/or acknowledging.

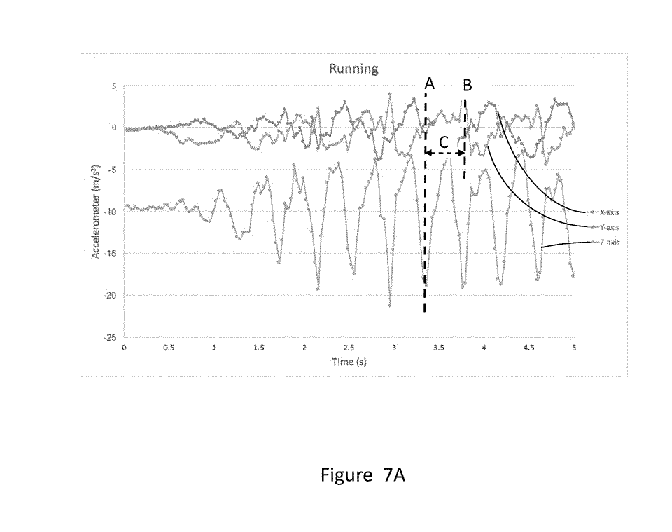

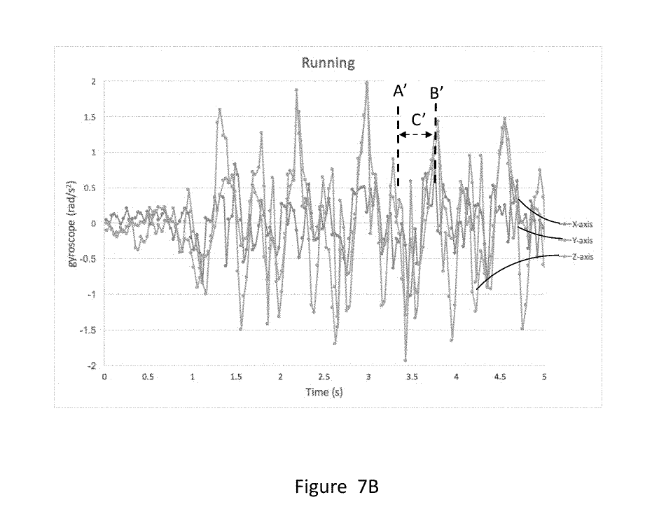

Returning to step 520 of FIG. 5, another example embodiment would identify the user movement as the fundamental movement of “running”, again by examining the data generated by at least one sensor in each of the user's shoes, and wirelessly transmitted to the processing device, using a technology such as BTLE. In the case of accelerometer sensors, outputting data indicative of ground reaction force, data such as those shown in FIGS. 7A and 7B may be obtained.

FIG. 7A shows measured three-axis data obtained from an accelerometer in one shoe. Experience has shown that the largest y-axis peaks B correspond to heel strikes, and the secondary y-axis peaks A correspond to toe-off, the interval C between A and B being the swing phase. In this example, the processor can determine that the user is running, not walking, by comparing the sensor data from two sensors (e.g., one corresponding to the left foot and one corresponding to the right foot). In this case, there are time intervals during which one foot indicates a toe-off event before the other foot has a heel strike event. This indicates that there are time intervals where neither foot is in contact with the ground, which is indicative of running. Other indications of the fundamental movement of running may include the leg swing velocity during the swing phase being above a particular threshold, or the ratio of leg swing velocity for one leg to the velocity of the stance leg exceeding a particular value. Velocity in the latter cases may readily be derived from accelerometer data.

FIG. 7B shows measured three axis-data obtained from a gyrometer in one shoe, again providing data on rotational force (derived from angular rotation about each axis) as a function of time. Features A′, B′, and C′ correspond in time to features A, B, and C in FIG. 7A, with the largest peak clusters B′ indicating heel strike, and the smaller intervening peak clusters A′ indicating toe-off. Although not shown in FIG. 7B, a strong peak in magnitude outside a defined norm in the gyrometer axis at the point of toe-off may indicate, for example, risk for ankle sprain, loss of energy propulsion to leave the ground, and/or poor transition to swing phase or gait. Further, repeated peaks (e.g., 3 or more times in 10 seconds) may increase the risk factor and/or alter the suspected diagnosis; for example, the posterior tibialis muscle may be weak.

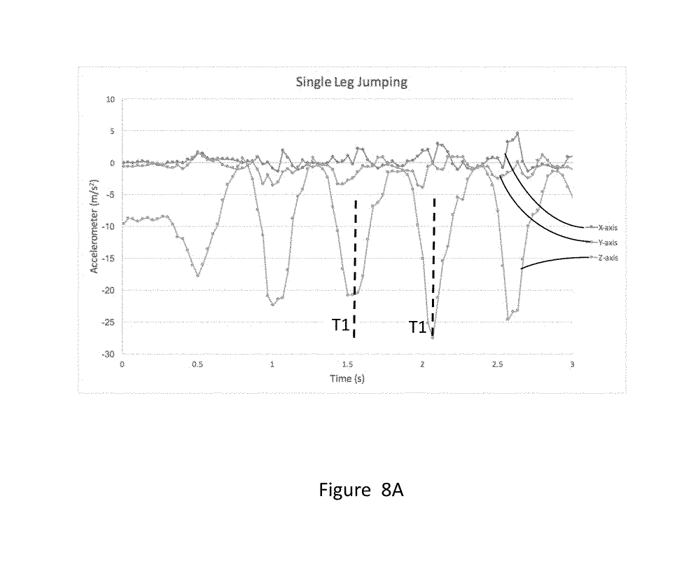

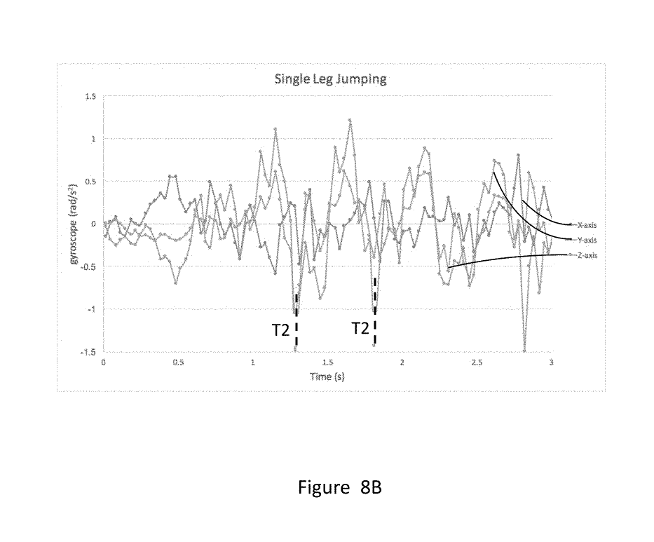

Returning to step 520 of FIG. 5, another example embodiment would identify the user movement as the fundamental movement of “single leg jumping”. In this case, data from a sensor in one shoe—the shoe of the jumping leg—can show the features that characterize single leg jumping. FIG. 8A shows measured three-axis data obtained from an accelerometer in one shoe. The z-axis trace shows deep troughs T1 corresponding to loading, when the foot lands on the ground, while the x and y traces are tightly bundled together. Three-axis data obtained from a gyrometer in one shoe for this same example show sporadic waveforms (including, for example troughs T2) in the y-axis traces. This may indicate imbalance, poor coordination, random take-off and landing and so on. An example response may include notifying the user to slow down the frequency of jumping, shorten the jump height, and/or use handhold assist to improve balance.

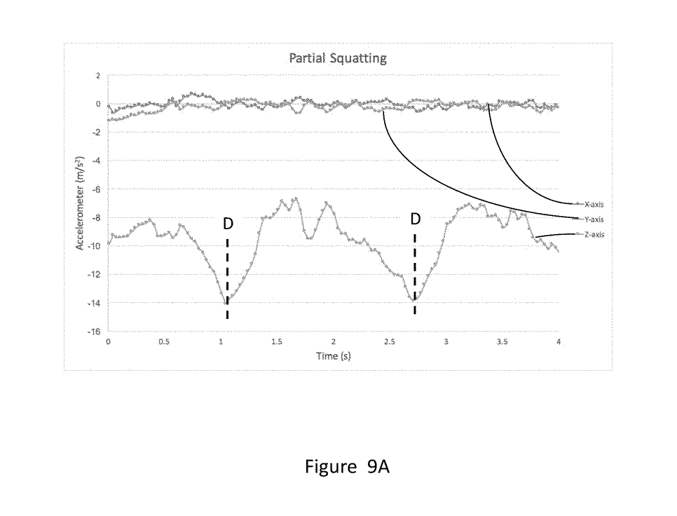

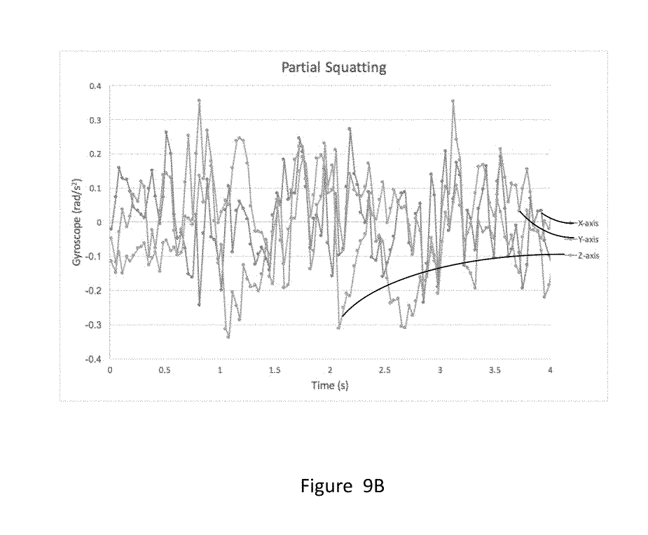

Returning to step 520 of FIG. 5, another example embodiment would identify the user movement as the fundamental movement of “squatting.” In this case, data are usefully gathered by one or more inertial movement sensors in each shoe. FIG. 9A shows measured three-axis data obtained from an accelerometer in one shoe sensor, indicating ground reaction forces as a function of time; an accelerometer in the other shoe sensor would yield the same data pattern. The x-axis and y-axis traces are tightly bundled together, and the z-axis trace shows very clear troughs D, corresponding to the drop phase of the squatting movement. In this particular case, the movement is not actually a full squat, where the knees are maximally bent so that the user's bottom is very close to the heels, but a partial squat, where the knees are bent to approximately 60 to 90 degrees, and the user's bottom is approximately half way down towards the heels. FIG. 9B shows corresponding data obtained from a gyrometer sensor in one shoe for the same sort of partial squat movement. As can be seen by examining the y-axis trace in FIG. 9B, the amplitude of the drop phase of the second partial squat is shallower than the first partial squat, showing an inconsistency in the movement. This could be, for example, the result of fatigue and/or pain experienced by the user, which may be further identified in subsequent movements (not shown). For example, if the corresponding data in the x-axis and z-axis diverge from a normal frequency during the squat movement, this would be indicative of a weight shift off of center. Depending on if this occurs in the drop-phase or rise-phase of the squat (identified by the leading or trailing portion of the trough D, respectively), it would have different clinical implications. For example, such divergence on the drop-phase and not on the rise-phase indicates eccentric quadricep weakness or inactivation. Alternatively, divergence on the rise-phase and not on the drop-phase may imply fear avoidance of pain.

Returning to step 520 of FIG. 5, another example embodiment would identify the user movement as the fundamental movement of “direction change”, also known as shuffle direction change. The directional information in the inertial sensors' outputs is particularly useful in identifying this movement, as all the sensors (accelerometers, gyrometers and magnetometers) are sensitive to ground reaction force as a function of time, with gyrometers clearly indicating changes in the plane of the foot, while magnetometers provide an absolute direction reference.

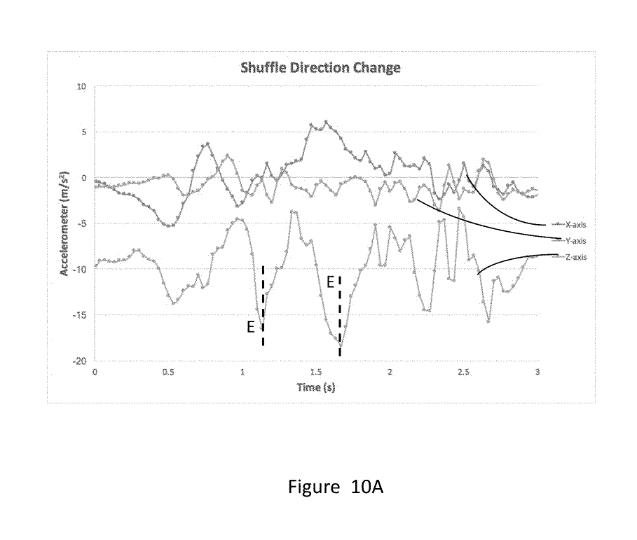

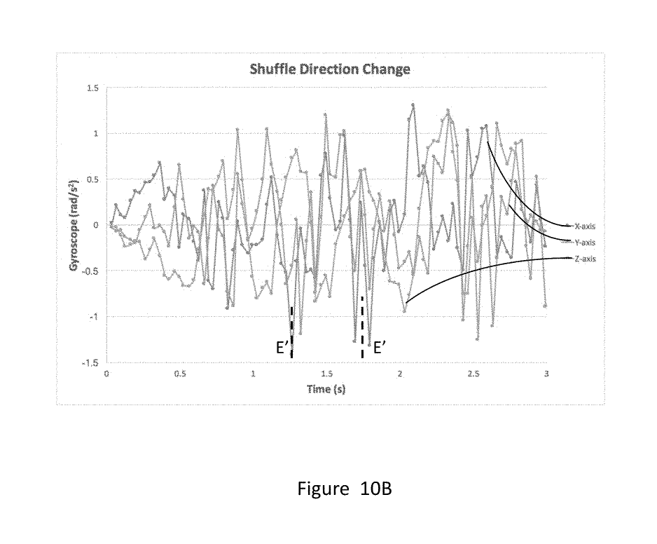

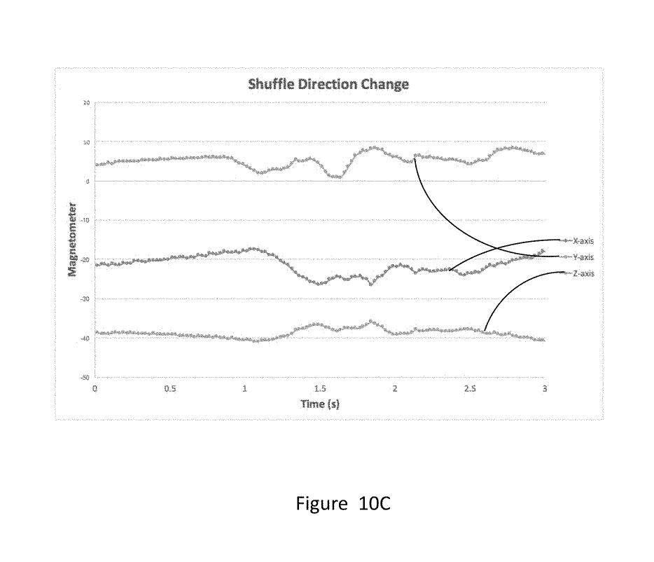

FIG. 10A shows accelerometer data traces obtained from an accelerometer in one shoe, where troughs E occur in the z-axis trace, at points where the foot direction changes. Corresponding measured gyrometer data (shown in FIG. 10B) exhibit deep troughs E′ in the x-axis trace, again indicating points of direction change. FIG. 10C shows data obtained from a magnetometer in the shoe for the same movement. Note that changes in all axis (x, y, and z) of the magnetometer diverge from the normal trajectory corresponding to direction changes in the shuffle movement. Normally, a more significant magnitude change could be identified for the outside planting foot, and If a more significant difference was not identified between the two feet, then the user might be landing too soft or too weak, not making a hard cut, and so on giving rise to an indication of sub-optimal performance.

### Examples of Analyzing Movement by Comparison With Pre-Established Signatures

The following examples concern steps 530 and 540 of FIG. 5. In each case, the data shown are obtained from insole-positioned sensor devices including a 3-axis accelerometer and a 3-axis gyrometer. In some embodiments, data may be obtained from a magnetometer in addition to or instead of the accelerometer or gyrometer. In general, analysis of the data obtained from a sensor device in just one foot may provide results of clinical importance, but there is often additional value in comparing data from both feet, focusing on differences between corresponding traces for each foot. As noted above, the processing device may assign a timestamp to each series of data received from each sensor when it is received at the processing device, to enable accurate time-based comparisons to be made between data from two or more independent sensors, for example from one sensor in each shoe.

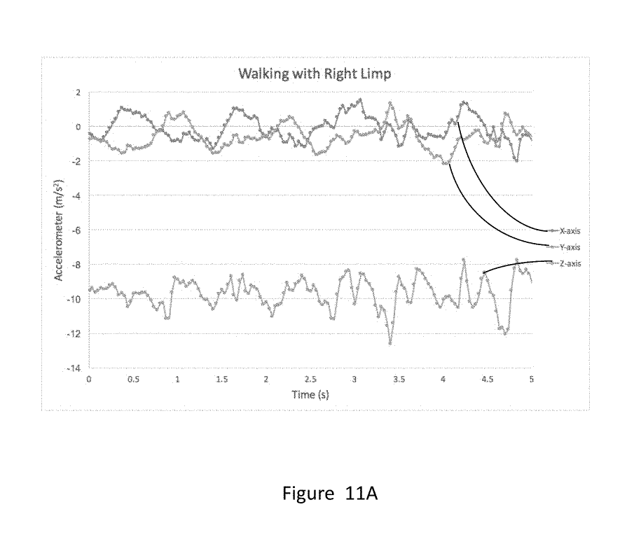

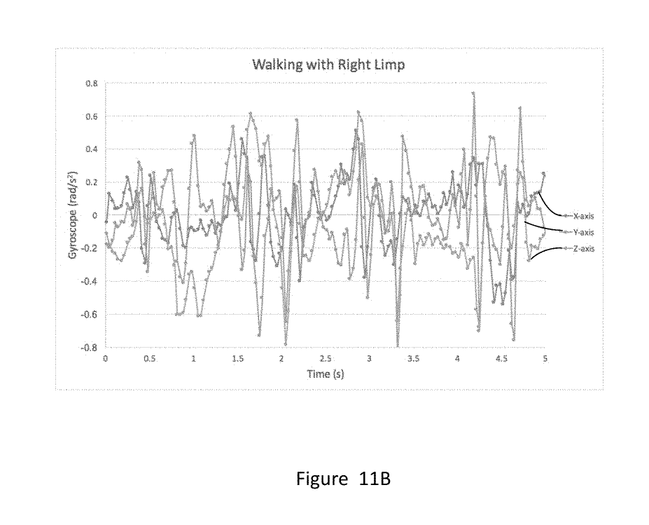

A first example of movement analysis is illustrated in FIGS. 11A and 11B, where the three-axis data are obtained from an accelerometer in FIG. 11A, and from a gyrometer in FIG. 11B, each included in a sensor device positioned in the right insole of a user walking with a right limp. Comparing these traces with pre-established signatures for normal walking, the feature that allows the processor to determine that this user is limping in the case of FIG. 11A is the significant phase shift between corresponding peaks of traces obtained from the x-axis sensor and the y-axis sensor. In the case of FIG. 11B, the features indicating limping are the clusters of large swings in magnitude in the data from both x-axis and y-axis gyrometer sensors.

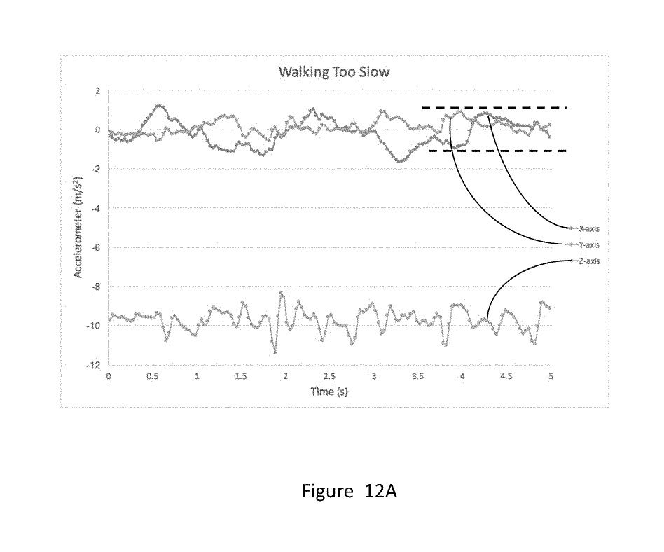

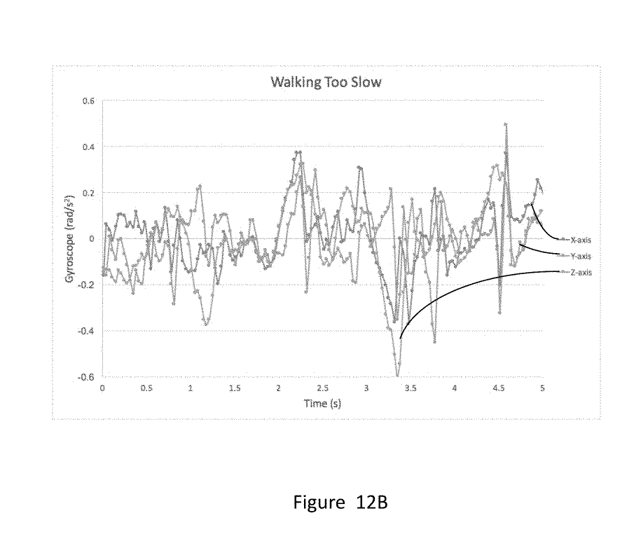

A second example is illustrated in FIGS. 12A and 12B, showing accelerometer and gyrometer data respectively, for a case where the user is walking too slowly than is considered desirable. This may be an indication that the user is at a high risk for falling, has hip arthritis, etc. Comparing the measured data in these traces with pre-established signatures for “normal” walking, as defined for the individual user, the accelerometer traces show abnormally small magnitudes, and the gyrometer traces show a lot of peak magnitude variability, suggesting the user is experiencing twisting or rolling instabilities about each axis of measurement.

Several of the embodiments discussed above so far have concerned analysis of complete fundamental movements. Embodiments herein also enable the analysis of specific phases of fundamental movements.

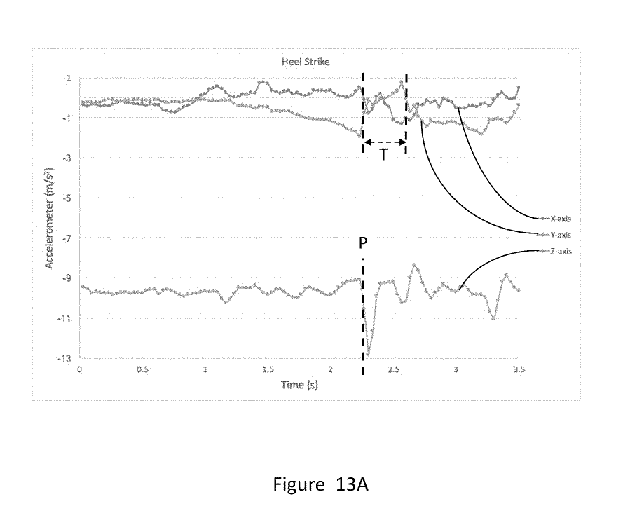

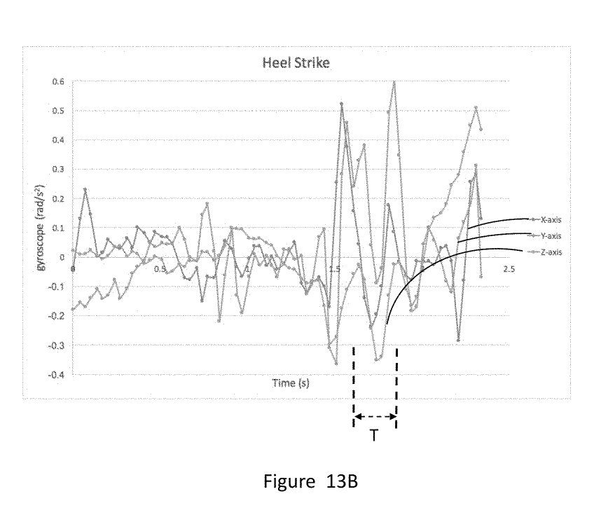

One such example is illustrated in FIGS. 13A and 13B. The phase of interest here is the heel strike phase or “stance” phase, involving sub-talar and mid-foot motion. The three axis accelerometer traces showing ground reaction force data (see FIG. 13A) show the point P of initial contact of the heel with the ground and the stance phase T, during which the foot remains in contact with the ground. The gyrometer traces indicative of direction change (see FIG. 13B) also show stance phase T, the data gathered within this time interval reflecting the kinematics of the foot between heel strike and toe-off, and providing information on whether over-pronation is occurring. For example, over-pronation would be occurring if the gyrometer data for x-axis, z-axis, (and possibly) y-axis significantly deviate from their normal walking signature. If over-pronation was detected, one possible notification/solution would be to increases the firmness of the orthotic at the midfoot region (essentially limiting or decreasing the amount of foot pronation).

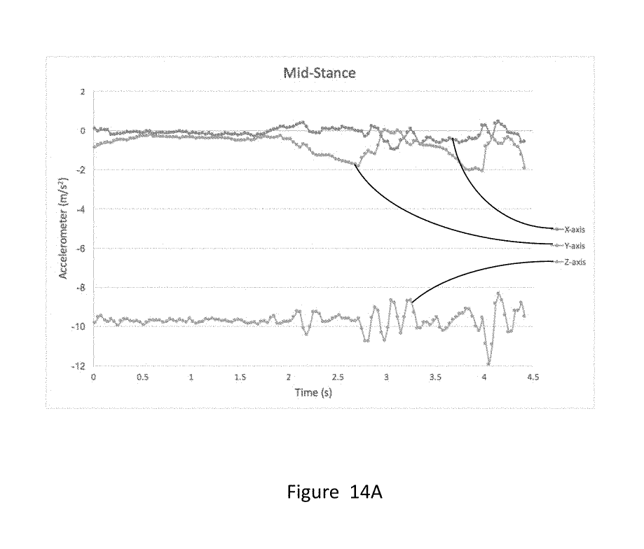

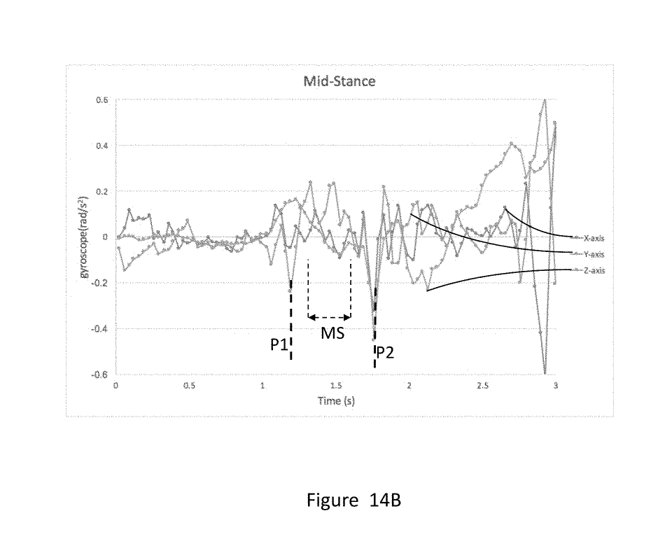

Another example is illustrated in FIGS. 14A and 14B, for a mid-stance phase, involving mid-tarsal motion. The gyrometer data of FIG. 14B is particularly informative in this case, showing features corresponding to heel strike (P1) and toe-off (P2), bounding the mid-stance period, MS. In this example, the mid-stance period between the heel strike and toe-off shows increased gyrometer activity which may represent foot pronation greater than what is typically present at the instantaneous heel strike and toe-off.

The kinetic and/or kinematic movements may be determined by analyzing data corresponding to fundamental movement phases, such determination being particularly useful in identifying problems of instability, asymmetry, and inefficiency. Comparison of traces with corresponding traces recorded from a “normal” or other reference population, or from the same user at a previous time may be particularly instructive. In some cases, an appropriate response to the detection of such problems is for the processing device to alert the user that a physical examination by a health professional might be beneficial. In other cases, an appropriate response may be for the processing device to simply notify the user that conscious attention should be paid to improving stance or gait as previously taught or advised. In some cases, an appropriate response may be to suggest adjusting a setting on an aid to improved stance or gait, for example to adjust an orthotic device, such as insole 400, by dialing in a different stiffness setting on dial 405. In other cases, the orthotic device may be directly controlled by the processor, without requiring direct input from the user.

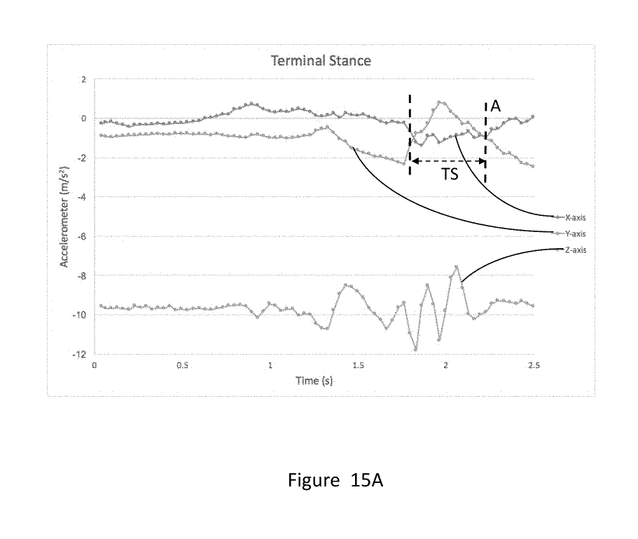

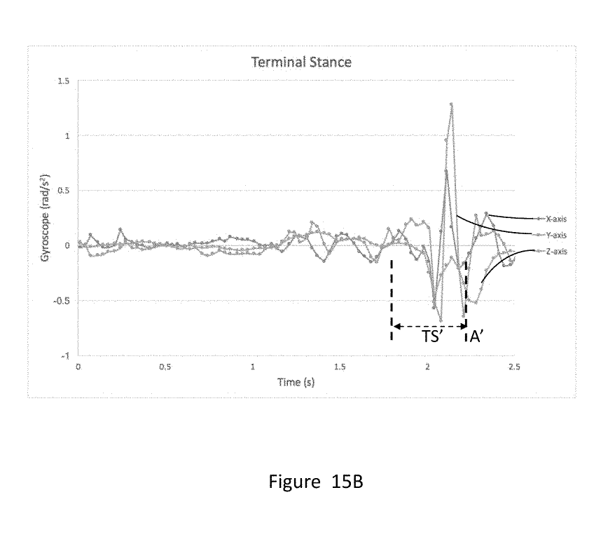

One example of detecting kinetic problems is illustrated in FIGS. 15A and 15B, for a terminal stance phase, involving the mechanics of forefoot stability. The accelerometer data of FIG. 15A show toe-off point A marking the end of terminal stance phase TS. Corresponding gyrometer data in FIG. 15B show strong activity in x-axis and y-axis traces during terminal phase TS′ culminating at toe-off point A′, after which stability is restored.

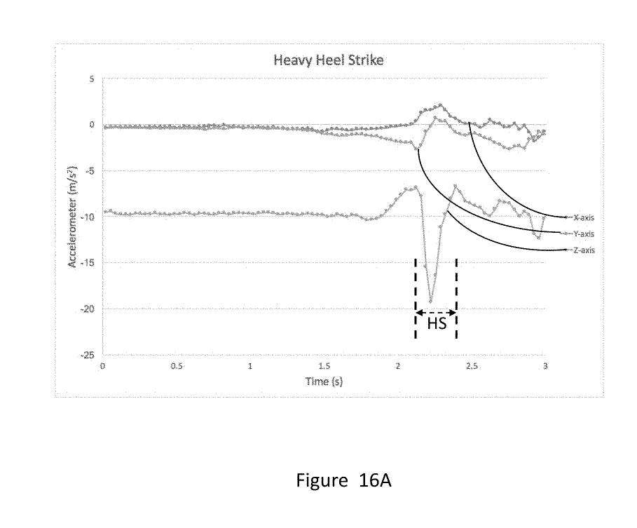

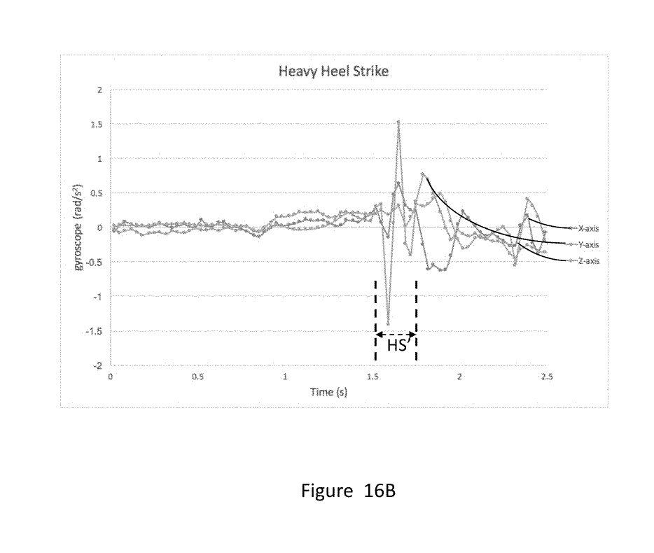

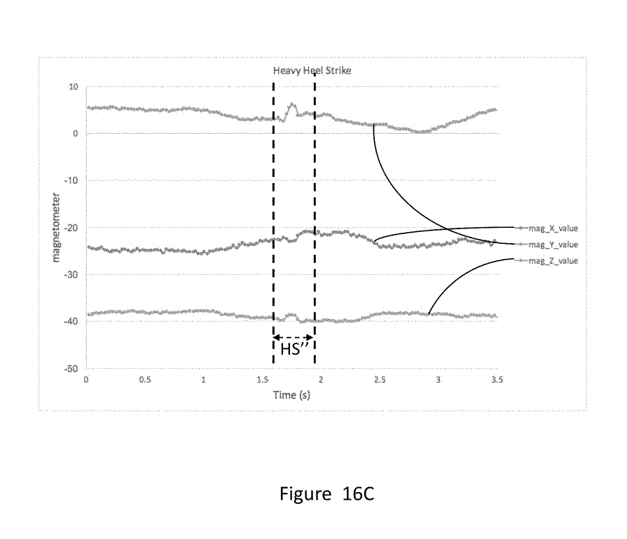

Another example is illustrated in FIGS. 16A, 16B and 16C for a heel strike phase (HS, HS′ and HS″ respectively), where data regarding ground force magnitude and direction can yield useful insights on whether step length and stride length are appropriate for the individual user (depending on their height and/or leg length for example), and on whether differences between data gathered from each foot suggest leg length discrepancies (predictive of increased chance of developing degenerative hip and knee joints due to higher ground reaction forces). Identification of this sort of problem from data analysis may be followed by providing an alert to the user to seek medical guidance, as discussed further below.

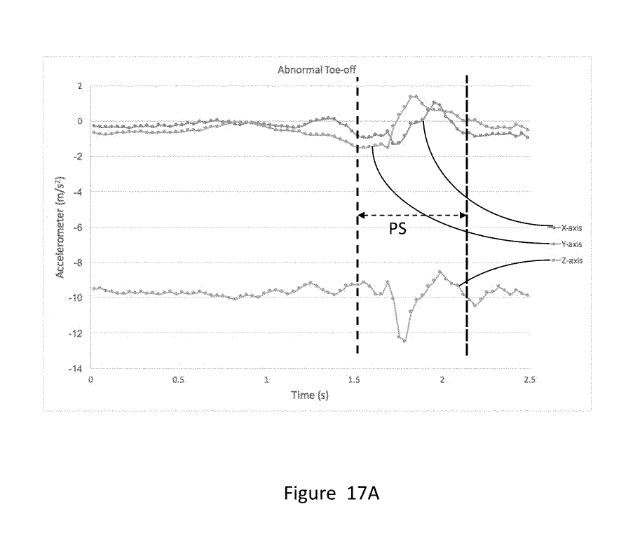

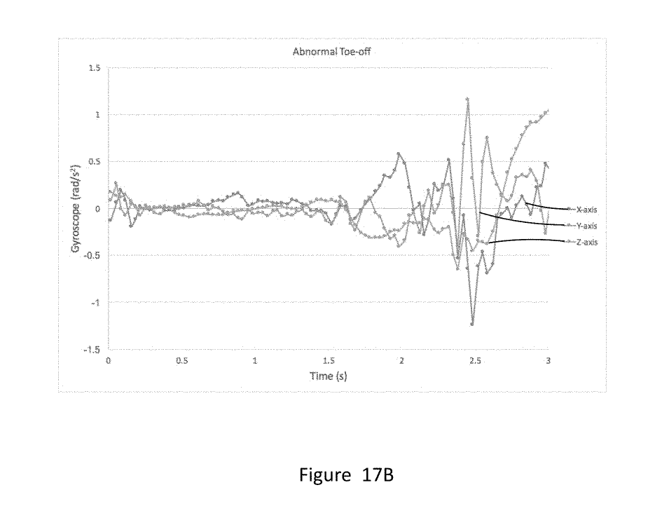

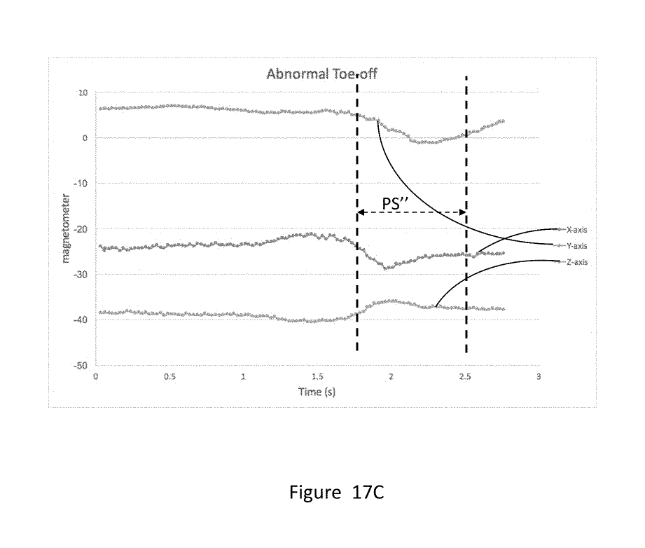

Yet another example is illustrated in FIGS. 17A, 17B and 17C for a toe-off or pre-swing phase, where features indicating an abnormally long duration (PS in FIG. 17A, PS″ in FIG. 17C) and irregular waveform activity axis activity (see FIG. 17B) are suggestive of undue forefoot stress and inefficient limb swing.

One more example involves the use of such data analysis to detect “near-injury” events, such as ankle rolling, track their incidence over time, and send the user notification if particular thresholds of incidence or rates of increase in incidence are crossed. For example, a user might have three near-ankle rolls in a typical run, but if the processing device detects twenty such incidents, a notification to the user is probably warranted. Similarly, if the user is undergoing rehab, storing data on the number and type of specific events such as “inversion moments” detected since the user's previous appointment with a therapist, and providing that data to the therapist at the next appointment, could clearly be helpful. Although not shown in the figures, erratic and abnormal data corresponding in time from both the accelerometer and gyrometer may indicate an ankle roll has occurred. Further, such data for a prolonged period of time may indicate a stumble or fall in addition to rolling of the ankle.

Features of the measured movement data that can be usefully compared with features of pre-established signatures include, but are not limited to, the duration of a particular phase, the onset timing of a phase determined from one axis relative to another, the timing of a phase or event for one limb compared to another, the peak to peak amplitude of a signal trace, the magnitude of a particular signal peak, timing of one phase relative to other phases, and the appearance or disappearance of particular peak clusters. As noted above, the term “characteristic” is defined to apply to the case where a feature of interest in the measured data matches a corresponding feature in the corresponding signature within a pre-established range. An alternative term such as “correct”, “normal”, or “typical” may in some cases be more appropriate. The term “uncharacteristic” is similarly defined to apply to any case where a feature of interest in the measured data falls outside a pre-established range for the corresponding feature in the corresponding signature. If, for example, the user baseline signature for the fundamental movement of walking shows a swing phase of 0.75 seconds, and the pre-established range is 0.65 seconds to 0.85 seconds, then if the processing device determines from newly gathered data that the swing phase is 1.05 seconds, the current swing phase would be considered uncharacteristic. An alternative term such as “incorrect” or “atypical” may be used rather than “uncharacteristic”.

In some embodiments, rather than a pre-established threshold range for a feature of interest, a single threshold value may be specified e.g. a swing phase value of 0.85 seconds may be specified as the maximum value that separates characteristic from uncharacteristic, without any minimum value being specified. Of course, this may also be taken as implying a range of 0 to 0.85 seconds.

### VII. Notifications

Following data analysis, comparing gathered data with signatures, notification of whether the analyzed movement is characteristic or not may be provided to the user in any of a variety of ways including but not limited to:

1) Visual notification via a display on the processing device or elsewhere. Some examples include:
a) Different color (e.g. red, yellow, and green) blocks displayed on a display screen, coded according to status, with one indicating a corrective action to be taken, such as, for example, turning the dial of an orthotic to increase/decrease the arch.
b) Playback of a video stream, showing user motion captured via a video camera, with automated corrections overlaid on the screen; and/or showing show how the motion could be corrected; and/or showing correct motion with emphasis on the necessary corrections.

2) Audible notification via a speaker on the processing device or elsewhere, e.g., into open air or via ear-buds
a) Spectrum of audio that changes during the movement to provides tones guiding the user to modify their motion.
b) Audible commands suggesting correction.

3) Alert notification when the results indicate serious consideration or action is necessary.
a) If a user's issue (i.e., poor mechanics) is less problematic at 5 k steps, but will become more of an issue when the user walks 20 k steps, the processing device may alert the user (e.g., “danger zone”) as the number of steps approaches the higher number.
b) When the threshold of dysfunction meets a certain level, the processing device may alert the user with a notification (i) indicating they should seek a professional for assistance and/or (ii) instructing them with exercise solutions and/or corrective action.

### Examples of Use Scenarios

1. In one example, a person with one or more sensor device(s) placed in an insole in their shoe(s) begins walking. The user looks at their smartphone device and sees an indication that their fundamental movement is “walking” and that the orthotic insole is adjusted correctly for their gait; see FIG. 6 for examples showing how “walking” may be identified using data from the inertial sensor(s). At some point, the user begins to run and their gait changes; see FIG. 7 for examples showing how “running” may be identified using data from the inertial sensor(s). The user receives a notification on their smartphone device that their fundamental movement has changed (e.g., the fundamental movement has been identified as changing from walking to running), gait is incorrect (if indeed there is abnormal data compared to pre-existing ‘norms’ identified in the signature data for that movement), and that a change in the orthotic insole could improve their gait while running. A suggestion for adjustment might be given by the device. The adjustment might improve the gait overall when taking into account both walking and running, or running itself.

2. In another example, a person with one or more sensor devices placed in an insole in their shoe(s) begins walking. The user receives a notification on their smartwatch that their gait is incorrect and a change in the orthotic insole could improve their gait. For example, referring back to FIG. 11 where a limp has been identified, the notification may include a prompt asking the user if they would like to change the orthotic insole according to a suggested improvement. In an embodiment, the user manually changes the orthotic insole. In another embodiment, the user selects a choice to change the orthotic insole and a message is transmitted from the smartwatch to the orthotic insole to automatically make the change.

3. In another example, a person with one or more sensor devices placed in an insole in their shoe(s) is recovering from an injury and is being treated by a physical therapist. In order for the insurance company to continue treatment, the user must show signs of improvement. A baseline is set up for the individual, and measurements are uploaded to a server such that the insurance company can verify that the patient is making improvements over the baseline.

4. In another example, a person with one or more sensor devices placed in an insole in their shoe(s) is recovering from hip surgery. The clinician has asked to receive notification if the patient's gait changes such that they are favoring the new hip. If this incorrect motion is detected, then a notification is sent directly to the clinician.

5. In another example, a person with one or more sensor devices placed in an insole in their shoe(s) is working in a warehouse moving products. The Safety Board at the workplace is concerned with the safety of the workers and is passively monitoring the workers movement/load such as to identify events that may lead up to an accident. The Safety Board receives a notification that a worker is carrying a load that is too heavy or is starting to show signs of fatigue. The Safety Board can proactively assist the worker before any damage is done to the worker.

Embodiments described herein provide various benefits. More specifically, embodiments allow for the convenient gathering and analysis of data indicative of user movement, such data being useful for many purposes, including clinical decision making, biofeedback or other patient learning, and for documenting progress for review by medical insurers. Some embodiments are particularly directed to understanding the mechanics of a particular part of the body, such as the foot, ankle, knee etc.

Embodiments may be implemented by using a non-transitory storage medium storing instructions executable by one or more processors to facilitate data entry by carrying out any of the methods described herein.

The above-described embodiments should be considered as examples, rather than as limiting the scope of the invention. Various modifications of the above-described embodiments will become apparent to those skilled in the art from the foregoing description and accompanying drawings.

Additionally, references herein to “embodiment” means that a particular feature, structure, or characteristic described in connection with the embodiment can be included in at least one example embodiment of an invention. The appearances of this phrase in various places in the specification are not necessarily all referring to the same embodiment, nor are separate or alternative embodiments mutually exclusive of other embodiments. As such, the embodiments described herein, explicitly and implicitly understood by one skilled in the art, can be combined with other embodiments.

1. A method comprising:
receiving, by a processing device, first data sent over a first wireless communication channel from a first inertial sensor positioned in or on a first shoe worn by a user, wherein the first data are generated when the user performs a fundamental movement;
identifying, by the processing device, a first phase of the fundamental movement by finding a match, within a first tolerance, between a portion of the first data and data characteristic of the first phase;
comparing, by the processing device, a feature from the portion of the first data to a corresponding feature from a pre-established signature associated with the first phase; and
when the comparison yields a result that falls outside a pre-established threshold range, causing the processing device to display an indication that the feature from the portion of the first data is uncharacteristic.

2. The method of claim 1, further comprising:
identifying, by the processing device, a type of the fundamental movement by analyzing the first data.

3. The method of claim 1, further comprising:
receiving, by the processing device, an input that sets a type of the fundamental movement.

4. The method of claim 1 further comprising:
causing the processing device to provide a proposed adjustment to an orthotic worn by the user.

5. The method of claim 1, wherein the pre-established signature is established from data previously gathered from one or more subjects.

6. The method of claim 1, wherein the pre-established signature is established from data previously gathered from the user, and wherein the pre-established signature comprises a baseline signature for the user.

7. The method of claim 1, wherein the first inertial movement sensor is selected from the group consisting of accelerometers, gyrometers, and magnetometers.

8. The method of claim 1, further comprising:
receiving, by the processing device, second data sent over a second wireless communication channel from a second inertial sensor positioned in or on a second shoe worn by the user, wherein the second data are generated while the user performs the fundamental movement;
identifying, by the processing device, a second phase of the fundamental movement by finding a match, within a second tolerance, between a portion of the second data to a portion of the pre-established signature;
comparing a second feature from the portion of the second data to a corresponding feature from a pre-established signature associated with the second phase; and
when the comparison yields a second result that falls outside a second pre-established threshold range, causing the processing device to display an indication that the feature from the portion of the second data is uncharacteristic.

9. The method of claim 8, further comprising:
identifying a type of the fundamental movement by analyzing the first data and the second data.

10. A non-transitory computer-readable medium containing instructions executable by one or more processors of a computer system to:
receive, by a processing device, first data sent over a first wireless communication channel from a first inertial sensor positioned in or on a first shoe worn by a user, wherein the first data are generated when the user performs a fundamental movement;
identify, by the processing device, a first phase of the fundamental movement by finding a match, within a first tolerance, between a portion of the first data and data characteristic of the first phase;
compare, by the processing device, a feature from the portion of the first data to a corresponding feature from a pre-established signature associated with the first phase; and
when the comparison yields a result that falls outside a pre-established threshold range, cause the processing device to display an indication that the feature from the portion of the first data is uncharacteristic.

11. The non-transitory computer-readable medium of claim 10, wherein the instructions are further executable to identify, by the processing device, a type of the fundamental movement by analyzing the first data.

12. The non-transitory computer-readable medium of claim 10, wherein the instructions are further executable to receive, by the processing device, an input that sets a type of the fundamental movement.

13. The non-transitory computer-readable medium of claim 10, wherein the instructions are further executable to cause the processing device to provide a proposed adjustment to an orthotic worn by the user.

14. The non-transitory computer-readable medium of claim 10, wherein the pre-established signature is established from data previously gathered from one or more subjects.

15. The non-transitory computer-readable medium of claim 10, wherein the pre-established signature is established from data previously gathered from the user, and wherein the pre-established signature comprises a baseline signature for the user.

16. The non-transitory computer-readable medium of claim 10, wherein the first inertial movement sensor is selected from the group consisting of accelerometers, gyrometers, and magnetometers.

17. The non-transitory computer-readable medium of claim 10, wherein the instructions are further executable to:
receive, by the processing device, second data sent over a second wireless communication channel from a second inertial sensor positioned in or on a first shoe worn by a user, wherein the second data are generated when the user performs the fundamental movement;
identify, by the processing device, a second phase of the fundamental movement by finding a match, within a second tolerance, between a portion of the second data and data characteristic of the second phase;
compare, by the processing device, a feature from the portion of the second data to a corresponding feature from a pre-established signature associated with the second phase; and
when the comparison yields a result that falls outside a second pre-established threshold range, cause the processing device to display an indication that the feature from the portion of the second data is uncharacteristic.

18. The non-transitory computer-readable medium of claim 17, wherein the instructions are further executable to identify a type of the fundamental movement by analyzing the first data and the second data.

19. A system comprising:
a first inertial sensor positioned in or on a first shoe worn by a user, the first inertial sensor being configured to:
generate first data when the user performs a fundamental movement; and
transmit the first data through a first wireless communication channel; and
a processing device configured to:
receive the first data through the first wireless communication channel;
identify a first phase of the fundamental movement by finding a match, within a first tolerance, between a portion of the first data and data characteristic of the first phase;
compare a feature from the portion of the first data to a corresponding feature from a pre-established signature associated with the first phase; and
when the comparison yields a result that falls outside a pre-established threshold range, cause the processing device to display an indication that the feature from the portion of the first data is uncharacteristic.

20. The system of claim 19 additionally comprising:
a second inertial sensor positioned in or on a second shoe worn by a user, the second inertial sensor being configured to:
generate second data when the user performs a fundamental movement; and
transmit the second data through a second wireless communication channel;
wherein the processing device is further configured to:
identify a second phase of the fundamental movement by finding a match, within a second tolerance, between a portion of the second data and data characteristic of the second phase;
compare a feature from the portion of the second data to a corresponding feature from a pre-established signature associated with the second phase; and
when the comparison yields a result that falls outside a second pre-established threshold range, cause the processing device to display an indication that the feature from the portion of the second data is uncharacteristic.
